(19) United States
(12) Patent Application Publication
(10) Pub. No.: US 2026/0285796 A1
(43) Pub. Date: Sep. 24, 2026

(54) CONVERSION OF A BIOMASS-DERIVED HYDROCARBON FEEDSTOCK INTO ACRYLATE SALTS

(71) Applicant: IFP Energies Nouvelles, Rueil-Malmaison (FR)

(72) Inventors: Catherine LAROCHE, Rueil-Malmaison Cedex (FR); Lionel MAGNA, Rueil-Malmaison Cedex (FR); Ludovic CHAHEN, Rueil-Malmaison Cedex (FR)

(73) Assignee: IFP Energies Nouvelles, Rueil-Malmaison (FR)

(21) Appl. No.: 19/140,161

(22) PCT Filed: Dec. 12, 2023
(86) PCT No.: PCT/EP2023/085337

§ 371 Date: Jun. 17, 2025

(30) Foreign Application Priority Data
Dec. 20, 2022 (FR) FR2213913

(51) Int. Cl. C07C 51/15; C07C 1/20; C07C 11/04; C07C 57/04; C12F 3/02; C12P 7/06; C12P 7/10

(52) U.S. Cl. CPC C07C 51/15; C07C 1/20; C07C 11/04; C07C 57/04; C12F 3/02; C12P 7/065; C12P 7/10; C12P 2201/00

(57) ABSTRACT

The invention concerns a method and a plant for converting biomass into acrylate salts.

The method successively involves:
a) a step of treating the biomass to produce ethanol and CO₂;
b) a step of dehydrating the ethanol obtained at the end of step a) to obtain ethylene;
c) a step of synthesizing acrylate salts from the ethylene obtained at the end of step b) and from the CO₂ obtained at the end of step a).

### TECHNICAL FIELD

The invention relates to a process for treating biomass, preferably lignocellulosic biomass, to produce acrylate salts from a single biobased carbon source while at the same time maximizing the carbon yield.

The lignocellulosic biomass fermentation process allows ethanol and CO₂ to be produced, which can advantageously be converted, relative to the prior art, into acrylate salts.

### PRIOR ART

Acrylate salts are acrylic acid derivatives and are used inter alia with acrylic acid to produce superadsorbent polymers. It is known to those skilled in the art that it is possible to synthesize acrylate salts from acrylic acid by adding a base, just as it is possible to synthesize acrylic acid from acrylate salts by adding an acid.

At present, acrylic acid is produced industrially at high temperature by oxidizing propylene in the presence of a heterogeneous catalyst. However, the relatively high price of propylene has a strong impact on the price of acrylic acid and several ways of synthesizing acrylic acid have been developed, notably starting with carbon monoxide. U.S. Pat. No. 3,023,237 relates to the Reppe process allowing acrylic acid to be synthesized from acetylene and carbon monoxide. Although this process has been performed industrially, the synthesis of acrylic acid via the oxidation of propylene remains more economically viable.

Another alternative explored is the synthesis of acrylic acid or derivatives thereof starting from carbon dioxide (CO₂). CO₂ has the advantage of being inexpensive and upgrading it in the form of high value-added products is an appealing approach for reducing its environmental impact.

Patent application US 2016/016 876 discloses a process for manufacturing acrylic acid using ethylene oxide and carbon monoxide via the formation of propiolactone. In certain embodiments, the propiolactone formation step is performed in the presence of ethylene oxide and CO₂.

In another approach, patent application US 2018/057 439 proposes to use CO₂ as a precursor of carbon monoxide in the context of the Reppe process mentioned above.

Finally, another alternative is to use CO₂ directly without going through the carbon monoxide reduction process. There are many documents relating to the synthesis of carboxylic acids or carboxylate salts from CO₂ and olefins, notably ethylene. By way of example, mention may be made of WO 2019/053 541, WO 2019/053 540, WO 2015/173 296, CN104418737 or CN105622400.

Ethylene may be produced from fossil resources via a steam cracking process, but also by dehydration of bioethanol. It is well known that bioethanol can be produced by the fermentation of sugars from various biomasses. However, fermentations to produce ethanol generally do not have good “carbon” yields because part of it is lost in the form of CO₂. Furthermore, ethanol produced from “first generation” (1G) biomass is in competition with the agrifood sector and losing a large part of this resource in the form of a greenhouse gas is even more problematic.

Finally, there are also other ways of synthesizing acrylic acid from lignocellulosic biomass, notably via the synthesis of lactic acid. Unlike the preceding routes, these do not use CO₂ or ethylene. The step of converting the biomass into lactic acid generally produces no CO₂, but the step of dehydrating the lactic acid into acrylic acid is far from trivial.

The invention is thus directed toward overcoming all the drawbacks mentioned above. More precisely, the aim of the invention is to develop a process for treating biomass, preferably “second generation” (2G) lignocellulosic biomass, to produce acrylate salts from ethanol and CO₂ produced from fermentation.

### OBJECTS OF THE INVENTION

In the context described previously, a first object of the present description is to overcome the problems of the prior art and to upgrade carbon, and in particular biobased carbon in the form of CO₂, into high value-added compounds, and in particular into acrylate salts. Specifically, the present invention relates to a process for producing acrylate salts according to an arrangement of steps, allowing biomass to be converted into ethanol and CO₂ and then these products to be converted into acrylate salts, using one or more of the following steps in addition to or instead of certain steps in conventional acrylate salt syntheses.

According to a first aspect, the present invention relates to a process for converting biomass into acrylate salts, which successively includes:
a) a step of treating the biomass to produce ethanol and carbon dioxide;
b) a step of dehydrating the ethanol obtained on conclusion of step a) to obtain ethylene;
c) a step of synthesizing acrylate salts from the ethylene obtained on conclusion of step b) and the carbon dioxide obtained on conclusion of step a) in the presence of a catalytic precursor and a solvent.

The invention is based on the upgrading of CO₂, which is a by-product formed during the step of fermenting biomass into ethanol, and possibly on the reuse of the water produced during the dehydration step in the other steps of the process according to the invention. The present invention thus presents a sequence of unit operations serving to maximize the carbon yield of the synthesis of acrylate salts from biomass, preferably lignocellulosic biomass, and even more preferentially “second generation” (2G) lignocellulosic biomass.

According to one or more embodiments, step a) comprises the following sub-steps:
a1) a step of pretreating biomass to obtain a pretreated substrate;
a2) a step of enzymatic or chemical hydrolysis of the pretreated substrate obtained on conclusion of step a1) to obtain an enzymatic or chemical hydrolysis must;
a3) a step of alcoholic fermentation of the enzymatic or chemical hydrolysis must obtained on conclusion of step a2) to obtain ethanol and carbon dioxide.

According to one or more embodiments, sub-step a1) is performed by steam explosion under acidic conditions at a temperature of between 150° C. and 250° C. and for a time of between 5 minutes and 30 minutes.

According to one or more embodiments, sub-step a2) is performed by enzymatic hydrolysis in the presence of Trichoderma reesei cellulases.

According to one or more embodiments, when step a2) is an enzymatic hydrolysis, steps a2) and a3) are performed simultaneously.

According to one or more embodiments, step b) comprises the following sub-steps:
b1) a step of vaporizing a vaporization feedstock comprising the ethanol obtained on conclusion of step a) in a heat exchanger, said vaporization feedstock being introduced into said vaporization step at a pressure of between 0.1 MPa and 2.5 MPa so as to produce a vaporized feedstock;
b2) a step of superheating said vaporized feedstock obtained on conclusion of step b1) so as to bring said vaporized feedstock to an inlet temperature compatible with the dehydration reaction temperature;
b3) a step of dehydrating said feedstock obtained from step b2) in at least one adiabatic reactor containing at least one dehydration catalyst and in which the dehydration reaction takes place, operating at an inlet temperature of between 350° C. and 550° C. and at an inlet pressure of between 0.3 MPa and 1.8 MPa.

According to one or more embodiments, step c) is performed at a temperature of between 105° C. and 170° C., and at a pressure of between 1 MPa and 10 MPa.

According to one or more embodiments, step c) is performed in the presence of a base chosen from alkanolates of secondary or tertiary alcohols.

According to one or more embodiments, step c) is performed in the presence of a catalytic precursor based on a metal complex of a transition metal chosen from nickel (0) and palladium (0) complexes.

According to one or more embodiments, step c) is performed in the presence of a solvent chosen from anisole, cyclohexylpyrrolidone, N,N-dibutylformamide and dimethylacetamide.

According to one or more embodiments, said process also comprises a step d) of liquid-liquid separation of the acrylate salt in the presence of a counter-solvent which is immiscible with the solvent used in step c).

According to one or more embodiments, said counter-solvent is water resulting at least partly from step b) of dehydration of the ethanol.

According to one or more embodiments, the biomass is a lignocellulosic biomass.

According to a second aspect, the present invention relates to a facility for converting biomass into acrylate salts that is capable of performing the process according to the invention, said facility comprising:
a first reaction section allowing ethanol and carbon dioxide to be produced from biomass;
a second reaction section for dehydration of the ethanol into ethylene; and
a third reaction section allowing acrylate salts to be produced from ethylene and carbon dioxide.

### LIST OF FIGURES

FIG. 1 shows a schematic representation of one embodiment of the process and facility according to the present invention, allowing acrylate salts to be produced from biomass, preferably lignocellulosic biomass.

### DESCRIPTION OF THE EMBODIMENTS

Embodiments of the process according to the first aspect of the invention and of the facility according to the second aspect of the invention will now be described in detail. In the detailed description that follows, numerous specific details are disclosed in order to provide a deeper understanding of the process and of the facility. However, it will be apparent to those skilled in the art that the process and the facility can be utilized without these specific details. In other cases, well-known features have not been described in detail in order to avoid unnecessarily complicating the description.

### Definitions

In the present patent application, the term “to comprise” is synonymous with (means the same thing as) “to include” and “to contain”, and is inclusive or open and does not exclude other elements which are not stated. It is understood that the term “to comprise” includes the exclusive and closed term “to consist of”. Moreover, in the present description, an effluent comprising essentially or solely compounds A corresponds to an effluent comprising at least 90% by weight, preferably at least 95% by weight, very preferably at least 99% by weight, of compounds A.

In the present patent application, the groups of chemical elements are given, by default, according to the CAS classification (CRC Handbook of Chemistry and Physics, published by CRC Press, Editor-in-Chief D. R. Lide, 81st edition, 2000-2001). For example, group VIII (or group VIIIB) according to the CAS classification corresponds to the metals from columns 8, 9 and 10 according to the new IUPAC classification; group VIB according to the CAS classification corresponds to the metals from column 6 according to the new IUPAC classification.

In the present patent application, “biomass” refers to any biologically produced feedstock, preferably produced by fermentation of sugars derived, for example, from sugar-yielding plant crops, such as sugar cane (saccharose, glucose, fructose and sucrose), from beetroot, or also from starchy plants (starch) or from lignocellulosic biomass or from hydrolyzed cellulose (glucose (predominantly) and xylose, galactose), containing variable amounts of water. Preferably, the biomass is a lignocellulosic biomass, and even more preferentially a “second generation” (2G) lignocellulosic biomass.

### DETAILED DESCRIPTION

The present invention may be defined as a process comprising a sequence of reaction steps making it possible to produce acrylate salts from biomass, preferably lignocellulosic biomass, and even more preferentially “second-generation” lignocellulosic biomass (2G). More particularly, the present invention relates to a process for converting biomass into acrylate salts, successively comprising:
a) a step of treating biomass, preferably lignocellulosic biomass, to produce ethanol and carbon dioxide (CO₂);
b) a step of dehydrating the ethanol obtained on conclusion of step a) to obtain ethylene;
c) a step of synthesizing acrylate salts from the ethylene obtained on conclusion of step b) and the CO₂ obtained on conclusion of step a).

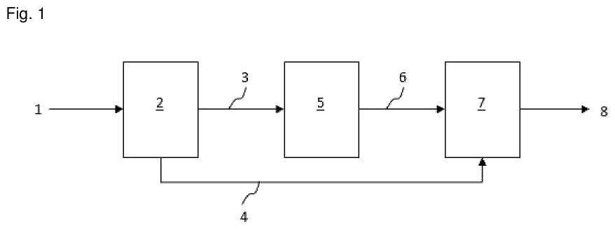

Moreover, the present invention can also be defined as a facility that is suitable for performing the process according to the invention, as illustrated in FIG. 1, said facility notably comprising:
a first reaction section 2 allowing ethanol 3 and CO₂ 4 to be produced from biomass 1;
a second reaction section 5 for dehydration of ethanol 3 into ethylene 6; and
a third reaction section 7 allowing acrylate salts 8 to be produced from the ethylene 6 and CO₂ 4.

### First Reaction Section (Step a) of the Process According to the Invention)

The first reaction section 2 allows ethanol 3 and CO₂ 4 to be produced from biomass 1.

In one embodiment according to the invention, the biomass used in the process is a lignocellulosic biomass, preferably a “second-generation” lignocellulosic biomass. Hardwood and cereal straw are the most commonly used substrates. Most of them consist of about 40% to 50% cellulose, 20% to 25% hemicellulose and 15% to 25% lignin. Other resources, dedicated forestry crops, residues from alcohol-yielding, sugar-yielding and cereal-yielding plants, residues from the paper industry and products from the processing of cellulose-based and lignocellulosic materials can be used.

In one embodiment according to the invention, the process for converting biomass into ethanol more particularly comprises the following sub-steps:
a1) a step of pretreating biomass, preferably lignocellulosic biomass, to obtain a pretreated substrate;
a2) a step of enzymatic or chemical hydrolysis of the pretreated substrate obtained on conclusion of step a1) to obtain an enzymatic or chemical hydrolysis must;
a3) a step of alcoholic fermentation of the enzymatic or chemical hydrolysis must obtained on conclusion of step a2) to obtain ethanol and CO₂.

Physicochemical pretreatment (step a1))

The pretreatment step a1) allows the production of a pretreated substrate comprising the sugars contained in the hemicelluloses in the form of monomers, essentially pentoses, such as xylose and arabinose, and hexoses, such as galactose, mannose and glucose, and improves the accessibility of the cellulose embedded in the lignin and hemicelluloses matrix.

A wide range of technologies are available, including acid cooking, alkaline cooking, steam explosion and organosolv pulping treatments. The effectiveness of the pretreatment is measured by the hemicellulose recovery content and by the susceptibility of the cellulose residue to hydrolysis. Acid pretreatments, under mild conditions, and by steam explosion, are the best suited as they allow total recovery of the pentoses and good accessibility of the cellulose to hydrolysis.

Preferentially, pretreatment step a1) is performed by steam explosion under acidic conditions at a temperature advantageously between 150° C. and 250° C. and for a time advantageously between 5 and 30 minutes. In this embodiment, step a1) allows the hemicelluloses to be converted into monomers while at the same time minimizing losses, notably of furfural, xylose being the predominant sugar. The sugars released are then extracted by washing in the aqueous phase. The solid residue (i.e. the pretreated substrate also referred to here as the cellulose-based residue) obtained on conclusion of the extraction then contains only cellulose and lignin.

Enzymatic or Chemical Hydrolysis (Step a2))

The pretreated substrate obtained on conclusion of step a1) is then hydrolysed, either acidically (i.e. chemically) or enzymatically using cellulolytic and/or hemicellulolytic enzymes. Microorganisms, such as fungi belonging to the genera Trichoderma, Aspergillus, Penicillium or Schizophyllum, or anaerobic bacteria belonging, for example, to the genus Clostridium, produce these enzymes, containing in particular cellulases and xylanases, suitable for the total hydrolysis of polymers constituting plants.

The acidic route, performed with strong acid, and more particularly with sulfuric acid, is effective but requires large amounts of chemical products (acid and then base for neutralization). Enzymatic hydrolysis does not have this drawback; moreover, it is performed under mild conditions and is effective.

In a preferential manner, the pretreated substrate, freed or not from the hydrolyzed hemicellulosic fraction and, where appropriate, the lignin, is hydrolyzed with the cellulolytic and/or hemicellulolytic enzymes produced by the specialized strains, Trichoderma reesei cellulases being the most effective and the most appropriate when the carbon-based substrates are derived from cellulosic or lignocellulosic biomass. The pretreated substrate to be hydrolyzed is preferably suspended in an aqueous phase in a proportion of 6% to 25% dry matter, preferably 10% to 20%, the pH is adjusted to between 4 and 5.5, preferably between 4.8 and 5.2 and the temperature to between 4° and 60° C., preferably between 45 and 50° C. The hydrolysis reaction is started by adding the cellulases; the amount usually used is from 10 mg to 30 mg of excreted protein per gram of pretreated substrate. The reaction generally lasts from 15 hours to 48 hours depending on the efficacy of the pretreatment, the composition of the cellulase mixture and the amount of enzymes added. The reaction is monitored by assaying the sugars released, notably glucose. The sugar solution (must) is then separated from the non-hydrolyzed solid fraction, essentially consisting of lignin, by filtration or centrifugation; this must is used for ethanol fermentation. When the cellulose fraction has been freed from the hydrolyzed hemicelluloses in the treatment step, glucose is the predominant sugar contained in the must.

Fermentation (Step a3))

Alcoholic fermentation is a biochemical process in which the sugars (carbohydrates, principally glucose) contained in the must are transformed into alcohol, preferentially ethanol, in a liquid medium deprived of air (anaerobic). The step of fermenting sugars to obtain ethanol is well known to those skilled in the art.

Alcoholic fermentation is preferentially performed at a temperature of between 25° C. and 32° C. For a more complete description of the conventional fermentation processes, reference may be made to the textbook “Les Biocarburants, État des lieux, perspectives et enjeux du développement [Biofuels, current state, perspectives and development challenges]”, Daniel Ballerini, published by Technip, 2006.

In general, ethanol is separated from the fermentation must by distillation and the residue is constituted of the distillation stillage. Regular or continuous distillation of the ethanol is necessary because above 14% ethanol, certain yeasts may be “poisoned”, resulting in a loss of productivity. Distillation is performed to allow production of an ethanol feedstock suitable for the dehydration process described later.

The CO₂ is recovered in the form of gas at the fermenter outlet. According to an essential aspect of the invention, the CO₂ obtained on conclusion of the fermentation step is sent at least partly to the acrylate salt synthesis step (step c) of the process according to the invention). The CO₂ may be compressed before storage or before use by means of compressors.

The ethanol fermentation residue, after separation from the ethanol, may be used as an inductive carbon source or as the main carbon source for enzyme production. The concentration of this residue is preferably adjusted to obtain the carbon source concentration best suited for the cellulolytic and/or hemicellulolytic enzyme production process.

The enzymatic hydrolysis and fermentation steps may be performed simultaneously (Simultaneous Saccharification and Fermentation (SSF) process), and then advantageously followed by a step of distillation and separation of the alcohol obtained.

### Second Reaction Section (Step b) of the Process According to the Invention)

The second reaction section 5 allows ethylene 6 to be produced from the ethanol 3 obtained from the first reaction section 2 (see FIG. 1).

The second reaction section allows the ethanol obtained on conclusion of step a) of the process according to the invention to be dehydrated to form ethylene. Ethanol dehydration is a process known to those skilled in the art, but optimizations are possible to reduce the energy cost of the process as described in patent application US 2013/190 547.

Advantageously, the ethanol feedstock used in step b) of the process according to the invention is a concentrated hydrated ethanol feedstock. The term “concentrated hydrated ethanol feedstock” means an ethanol feedstock comprising a mass percentage of ethanol of greater than or equal to 35% by weight. Preferably, said concentrated ethanol feedstock comprises a mass percentage of ethanol of between 35% and 99.9% by weight relative to the total weight of the feedstock. Preferably, said concentrated ethanol feedstock comprises a mass percentage of ethanol of between 35% and 96% by weight relative to the total weight of the feedstock. Said concentrated ethanol feedstock also advantageously comprise, in addition to water, a content of alcohols other than ethanol, for instance methanol, butanol and/or isopentanol, of less than 10% by weight, and preferably less than 5% by weight, a content of oxygen-based compounds other than alcohols, for instance ethers, acids, ketones, aldehydes and/or esters, advantageously less than 1% by weight, and a content of organic and inorganic nitrogen and sulfur advantageously less than 0.5% by weight, the weight percentages being expressed relative to the total mass of said feedstock.

In one embodiment according to the invention, step b) comprises the following sub-steps:
b1) a step of vaporizing a vaporization feedstock comprising ethanol obtained on conclusion of step a) in a heat exchanger, said vaporization feedstock being introduced into said vaporization step at a pressure of between 0.1 MPa and 2.5 MPa so as to produce a vaporized feedstock;
b2) a step of superheating said vaporized feedstock obtained on conclusion of said step b1) so as to bring it to an inlet temperature that is compatible with the dehydration reaction temperature;
b3) a step of dehydrating said feedstock obtained from step b2) in at least one adiabatic reactor containing at least one dehydration catalyst and in which the dehydration reaction takes place, operating at an inlet temperature of between 350° C. and 550° C. and at an inlet pressure of between 0.3 MPa and 1.8 MPa.

The ethanol feedstock used in the context of step b) of the process of the invention advantageously undergoes a pretreatment step prior to step b1) of vaporization of said feedstock. Said pretreatment step makes it possible to remove the impurities contained in said feedstock so as to limit the deactivation of the dehydration catalyst placed downstream, and in particular compounds containing nitrogen and compounds containing sulfur. The oxygen-based compounds present in said feedstock are not substantially removed.

Said step of pretreatment of the ethanol feedstock is advantageously performed by means known to those skilled in the art, such as the use of at least one resin, the adsorption of the impurities onto solids preferably at a temperature of between 20° C. and 60° C.; a sequence comprising a first step of hydrogenolysis operating at a temperature of between 20° C. and 80° C. followed by step of uptake on acidic solid at a temperature of between 20° C. and 80° C., and/or distillation. In the case of the use of at least one resin, said resin is preferably acidic and is used at an elevated temperature of between 70° C. and 200° C. Said resin may optionally be preceded by a basic resin.

In the case where the pretreatment step is performed by adsorption of the impurities onto solids, said solids are advantageously chosen from molecular sieves, activated carbon, alumina and zeolites.

Said step of pretreatment of the ethanol feedstock makes it possible to produce a purified ethanol cut in which the organic impurities have been removed, so as to obtain a purified feedstock meeting the level of impurities compatible with the dehydration catalyst.

### Vaporization of the Feedstock (Step b1)

The feedstock at least partly comprising the optionally pretreated ethanol obtained on conclusion of step a) of the process according to the invention is referred to as the vaporization feedstock. Said vaporization feedstock advantageously also comprises a stream of water recycled in accordance with recycling step b5) or a stream of water external to the process. In this case, the mass ratio of the water stream, whether recycled or external to the process, to the pretreated ethanol stream is advantageously between 1 and 4, with the aim of lowering the partial ethanol pressures in the dehydration reactor(s) and making the process more selective in terms of ethylene.

According to one embodiment of the invention, the process comprises a step b1) of vaporizing said vaporization feedstock so as to produce a vaporized feedstock. Said vaporization is performed by means of heat exchange in a heat exchanger with a heat source which may be a stream internal or external to the process, or by direct heating (for instance in a furnace) or any other technique known to those skilled in the art.

Said vaporization feedstock is introduced in said vaporization step b1) at a pressure of between 0.1 MPa and 2.5 MPa and at an inlet temperature of between 350° C. and 500° C.

### Optional Compression Step

In a preferred embodiment, said vaporized feedstock undergoes compression in a compression step so as to produce a compressed feedstock. Said compression step is advantageously performed in any type of compressor known to those skilled in the art. In particular, the compression step is advantageously performed in a compressor of the radial compressor type with integrated gearbox or in a compressor comprising one or more blowers with a radial impeller connected in series without intermediate cooling or in a compressor of the positive displacement type with or without lubrication.

The optional compression step allows a heat pump to be formed which is integrated into said process, using the streams from the process, allowing the vaporization feedstock from step b1) to be vaporized by heat exchange with the effluent from dehydration step b3).

In the case where the optional compression step is performed, said vaporization feedstock is introduced into said vaporization step b1) at a pressure of between 0.1 MPa and 1.4 MPa, preferentially between 0.2 MPa and 0.6 MPa.

The pressure of said feedstock compressed on conclusion of the optional compression step is advantageously between 0.3 MPa and 1.8 MPa, preferentially between 0.5 MPa and 1.3 MPa. The outlet pressure of said feedstock is sufficiently high for the condensation temperature of the effluent from the last reactor to be higher than the vaporization temperature of the feedstock entering step b1), which is a necessary condition for the feasibility of step b1).

### Superheating Step b2)

Said vaporized feedstock, optionally compressed, can be heated in a single-phase gas type exchanger, by virtue of heat exchange with any stream internal or external to the process, preferably by heat exchange with the effluent from the last adiabatic reactor of step b3). In said single-phase gas type exchanger, said feedstock, optionally compressed, is superheated. In the case where heat exchange is performed with the effluent coming, in the gaseous state, from the last adiabatic reactor of step b3), the latter is “desuperheated” without being condensed.

In the case where the optional compression step is performed, said single-phase gas exchanger is an exchanger of a technology known to those skilled in the art which allows the pressure drops to be minimized while at the same time having a large exchange surface area. This gas/gas exchange at low pressure results in a low density of flow of heat through the wall of the exchanger (low transfer coefficient), which makes it necessary to have a large exchange surface area. Furthermore, the loss of pressure must be minimized so as to limit the charge of the compressor of the optional compression step. For example, this exchanger may be a pressurized plate exchanger in a calender, of the Packinox® type supplied by Alphalaval®.

Said vaporized feedstock, optionally compressed, optionally heated in said exchanger of the single-phase gas type, is then introduced into superheating equipment, preferably a furnace, so as to bring it to an inlet temperature in at least one adiabatic reactor compatible with the temperature of the dehydration reaction.

### Dehydration Step b3)

According to one embodiment of the invention, said feedstock resulting from step b2) undergoes a dehydration step b3) in at least one adiabatic reactor containing at least one fixed bed of dehydration catalyst and in which the dehydration reaction takes place.

The dehydration step b3) is advantageously performed in one or two reactors.

In the case where step b3) is performed in a single adiabatic reactor, said compressed and optionally heated feedstock is advantageously introduced into said reactor at an inlet temperature of between 350° C. and 550° C. and preferably between 400° C. and 500° C., and at an inlet pressure of between 0.3 MPa and 1.8 MPa, and preferably between 0.4 MPa and 0.8 MPa.

The effluent from said adiabatic reactor in step b3) advantageously has a temperature of between 270° C. and 450° C. and preferably between 340° C. and 430° C., and an outlet pressure of between 0.2 MPa and 1.6 MPa and preferably between 0.3 MPa and 0.8 MPa.

In the case where step b3) is performed in two adiabatic reactors, said compressed and optionally heated feedstock is advantageously introduced into the first reactor at an inlet temperature of between 350° C. and 550° C. and preferably at a temperature of between 370° C. and 500° C., and at an inlet pressure of between 0.3 MPa and 1.8 MPa, and preferably between 0.4 MPa and 1.1 MPa.

The effluent from the first adiabatic reactor advantageously leaves said first reactor at a temperature of between 270° C. and 450° C. and preferably between 290° C. and 390° C., and at a pressure of between 0.3 MPa and 1.7 MPa and preferably between 0.3 MPa and 1.0 MPa.

Said effluent is then advantageously placed in a furnace so that the inlet temperature of said effluent into the second adiabatic reactor is between 350° C. and 550° C. and preferably between 400° C. and 500° C. Said effluent has a pressure on entering said second reactor advantageously of between 0.3 MPa and 1.7 MPa and preferably between 0.3 MPa and 0.9 MPa.

The effluent from the second adiabatic reactor leaves said second adiabatic reactor at a temperature advantageously between 270° C. and 450° C. and preferably between 340° C. and 430° C. The outlet pressure of said effluent from the second adiabatic reactor is advantageously between 0.2 MPa and 1.6 MPa and preferably between 0.3 MPa and 0.8 MPa.

The inlet temperature of the reactor(s) may advantageously be gradually increased to avoid deactivation of the dehydration catalyst.

The dehydration reaction which takes place in at least one adiabatic reactor of step b3) of the process advantageously operates at a weight hourly space velocity (WHSV) of between 0.1 and 20 h⁻¹ and preferably between 0.5 and 15 h⁻¹. The weight hourly space velocity is defined as the ratio of the mass flow rate of the pure ethanol feedstock to the mass of catalyst. The dehydration catalyst used in step b3) is a catalyst known to those skilled in the art.

Said catalyst may be an amorphous acid catalyst, a zeolite acid catalyst, a silica-alumina-based catalyst, an alumina-based catalyst or a silica-alumina-based catalyst.

Said catalyst is preferably an amorphous acid catalyst or a zeolite acid catalyst.

In the case where the dehydration catalyst used in step b3) is a zeolitic catalyst, said catalyst comprises at least one zeolite chosen from zeolites having at least pore apertures containing 8, 10 or 12 oxygen atoms (8 MR, 10 MR or 12 MR). Specifically, it is known to define the size of the pores of zeolites by the number of oxygen atoms forming the ring section of the channels of the zeolites, referred to as “member ring” or MR. Preferably, said zeolitic dehydration catalyst comprises at least one zeolite having a structural type chosen from the MFI, FAU, MOR, FER, SAPO, TON, CHA, EUO MEL and BEA structural types. Preferably, said zeolitic dehydration catalyst comprises a zeolite of MFI structural type and preferably a ZSM-5 zeolite.

The zeolite used in the dehydration catalyst used in step b3) of the process according to the invention may advantageously be modified by dealumination or desilication according to any dealumination or desilication method known to those skilled in the art.

The zeolite used in the dehydration catalyst used in step b3) of the process or the final catalyst may advantageously be modified with an agent having the property of weakening its overall acidity and improving its hydrothermal resistance properties. Preferably, said zeolite or said catalyst advantageously comprises phosphorus, preferably added in H₃PO₄ form, followed by steam treatment after neutralization of the excess acid with a basic precursor, for instance calcium. Preferably, said zeolite comprises a phosphorus content of between 1% and 4.5% by weight, preferably between 1.5% and 3.1% by weight relative to the total mass of the catalyst.

Preferably, the dehydration catalyst used in step b3) is a catalyst described in patent applications WO 2009/098 262, WO 2009/098 267, WO 2009/098 268 or WO 2009/098 269.

In the case where the dehydration catalyst used in step b3) is an amorphous acid catalyst, said catalyst comprises at least one porous refractory oxide chosen from alumina, alumina activated by a mineral acid deposition and silica alumina.

Said amorphous or zeolitic dehydration catalyst used in step b3) may advantageously also comprise at least one oxide-type matrix also known as a binder. According to the invention, the term “matrix” means an amorphous or crystallized matrix or one comprising amorphous and crystallized portions. Said matrix is advantageously selected from the elements of the group formed by clays (such as for example from natural clays such as kaolin or bentonite), magnesia, aluminas, silicas, silica-aluminas, aluminates, titanium oxide, boron oxide, zirconia, aluminum phosphates, titanium phosphates, zirconium phosphates and charcoal, used alone or as a mixture. Preferably, said matrix is selected from the elements of the group formed by aluminas, silicas and clays.

Said dehydration catalyst used in step b3) is advantageously shaped into the form of grains of various shapes and sizes. It is advantageously used in the form of cylindrical extrudates or polylobal extrudates such as bilobal, trilobal or polylobal extrudates of straight or twisted form, but may optionally be manufactured and used in the form of crushed powder, lozenges, rings, beads, wheels or spheres. Preferably, said catalyst is in the form of extrudates.

Said dehydration catalyst used in step b3) is advantageously used in at least one reactor, in a fixed bed or in a moving bed.

In step b3) of the process according to the invention, the catalysts used and the operating conditions are chosen in such a way as to maximize ethylene production. The overall dehydration reactions performed in step b3) of the process according to the invention are as follows:

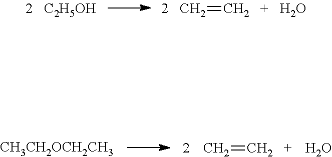

The conversion of the ethanol feedstock in step b) of the process according to the invention is greater than 90%, preferably 95% and more preferably greater than 99%.

A conversion of less than 90% has the effect of lowering the overall yield of the process, a larger amount of diethyl ether not converted into ethylene being lost in the downstream separation steps.

The conversion of the ethanol feedstock is defined, as a percentage, by the following mathematical formula:

$$[ 1 - ( mass per hour of ethanol leaving / mass per hour of ethanol entering ) ] \times 100$$

The mass per hour of ethanol entering and leaving is measured in a conventional manner, for example by chromatography.

Step b3) in which the dehydration reaction takes place is advantageously performed in one or two reactors. A preferred reactor is a radial reactor operating in an ascending or descending mode. During step b3) of the process according to the invention, the transformation of the feedstock is accompanied by the deactivation of the dehydration catalyst by coking and/or by adsorption of inhibitor compounds. The dehydration catalyst must thus periodically undergo a regeneration step. Preferably, the reactor is used in an alternating regeneration mode, also referred to as a swing reactor, in order to alternate the reaction and regeneration phases of said dehydration catalyst. The objective of this regeneration treatment is to burn the organic deposits and also the species containing nitrogen and sulfur present at the surface and within said dehydration catalyst. The optional pretreatment step makes it possible to reduce the amount of basic and organic impurities and also the cationic species which will alter the cycle time of the catalyst. The removal of these species thus makes it possible to limit the number of regenerations of the catalyst.

Optionally, for example in the case where the vaporization feedstock does not comprise any recycled water stream or any stream of water external to the process, the number of reactors can be increased in order to compensate for the endothermicity of the reaction by the presence of intermediate furnaces in the advancement of the ethanol dehydration reaction.

The effluent from the last adiabatic reactor of step b3) is optionally sent to a single-phase gas type exchanger in which it is “desuperheated” without being condensed by heat exchange with the compressed feedstock from the optional compression step, which, for its part, is superheated.

Said “desuperheated” effluent is then advantageously sent to a second gas/liquid type exchanger in which it is partially condensed by heat exchange serving to vaporize the vaporization feedstock.

### Separation Step b4) (Optional)

In one embodiment according to the invention, the effluent from step b3) undergoes a step b4) of separation into an effluent comprising ethylene at a pressure of less than 1 MPa and an effluent comprising water.

Step b4) of separation of said dehydration effluent resulting from step b3) may advantageously be performed by any method known to those skilled in the art, for instance by a gas/liquid separation zone, and preferably a gas/liquid separation column.

The effluent comprising ethylene at a pressure of less than 1 MPa then advantageously undergoes compression. Said compression makes it possible to raise the pressure of said effluent to a pressure advantageously of between 2 MPa and 4 MPa, necessary for its final purification.

Preferably, the effluent comprising ethylene separated on conclusion of step b4) is not recycled into at least one adiabatic reactor of step b3). The non-recycling of the ethylene separated on conclusion of step b4) in at least one adiabatic reactor of step b3) does not impair the ethylene selectivity of the process.

At least a portion of the effluent comprising water resulting from step b4) is optionally recycled into separation step b4). In the case where at least a portion of the effluent comprising water is recycled, said portion of the effluent comprising water is advantageously cooled with the aid of a cold fluid or a fluid resulting from the process and is preferably purified according to the known purification methods described below.

### Purification Step b5) (Optional)

In one embodiment according to the invention, at least a portion of the effluent comprising water resulting from the separation step b4) undergoes a purification step b5). The purification step b5) may advantageously be performed by any purification method known to those skilled in the art. By way of example, the purification step b5) may advantageously be performed by using ion exchange resins, molecular sieves, membranes, by adding chemical agents for adjusting the pH, for instance sodium hydroxide or amines and by adding chemical agents for stabilizing the products, for instance polymerization inhibitors chosen from bisulfites and surfactants.

At least one stream of purified water and at least one stream of unconverted ethanol are then separated. The separation may advantageously be implemented by any separation method known to those skilled in the art. By way of example, the separation may advantageously be performed by distillation, the use of molecular sieves, membranes, steam or heat stripping or by absorption in a solvent, for instance glycol solvents.

A stream containing the light gases, preferably acetaldehyde and methanol, may advantageously also be separated.

Use of the purified water stream from step b5) allows the vast majority of the ethylene to be separated from the water before it is recycled. The ethylene is thus separated from the diluent in the process according to the invention, allowing an inert thermal reaction diluent to be used for the process. Doing so also allows improved energy recovery, without degrading the final ethylene yield and selectivity.

### Third Reaction Section (Step c) of the Process According to the Invention)

The third reaction section 7 comprises at least one reactor in which the synthesis of acrylate salts is performed from the ethylene 6 obtained on conclusion of step b) and the CO₂ 4 obtained on conclusion of step a). In said reactor, the ethylene 6 from the second reaction section 5, the CO₂ 4 from the first reaction section 2, a catalytic precursor and a reaction solvent are placed in contact to form the active species. The term “active species” means the species formed when the catalytic precursor, the CO₂ and the ethylene are placed in contact. The synthesis of the acrylate salt is complete when the active species is placed in contact with a base. This base may be soluble or insoluble in the reaction solvent. The base may also be supported on a solid support. The base may already be present in the reactor during the placing in contact of the reagents in the reactor or it may be introduced in a step subsequent to the formation of the active species. Preferentially, the base is miscible with the solvent. In a more preferential manner, the base is introduced into the reactor at the same time as the other reagents.

### The Base

The base is generally an alkoxide salt such as a phenoxide salt or an alkanolate salt. In a more preferential manner, the base is chosen from alkanolates of secondary or tertiary alcohols (tert-butanol, isopropanol, etc.). The salts generally contain inorganic counterions such as Li, Na, Ca and Cs. The base counterion is that which will be associated with the final acrylate salt if no ion exchange step is envisaged. In a preferential manner, the bases will be sodium salts and the final acrylate will be a sodium acrylate.

### The Catalytic Precursor

As a general rule, the catalysts are transition metal complexes. Preferentially, nickel (0) and palladium (0) complexes are used in the examples. It is also possible to start from a nickel (2) or palladium (2) salt and reduce it in the presence of a reducing agent such as H₂, Mg, Na or Zn to form a nickel (0) or palladium (0) complex. In a preferential manner, Pd (0) is the preferred metal, preferentially in the form of [Pd(PPh₃)₄].

In general, the most active ligands appear to be polydentate ligands containing at least one phosphine coordinated to the metal center. In a more preferential manner, these are essentially bidentate ligands of the type (P,P); (P,N); (P,O); (P, carbene). In an even more preferential manner, the best results are obtained with (P,P) ligands preferentially 1,2-bis(dicyclohexylphosphino)ethane, 1,3-bis(dicyclohexylphosphino)propane and 1,4-bis(dicyclohexylphosphino)butane.

The catalytic precursor is generally a mixture of a metal precursor and one or more ligands. Preferentially, the catalytic precursor is a stoichiometric mixture of a bidentate ligand with a metal precursor. The metal precursor may be formed before the reaction (ex situ) or during the reaction (in situ) in the reaction solvent or in another solvent.

### The Solvent

The solvent may be selected from aromatics, halogenated aromatic compounds, ethers, alcohols, amides and ureas. The solvent is preferentially chosen from anisole, cyclohexylpyrrolidone, N,N-dibutylformamide and dimethylacetamide.

The reaction is generally performed with a total pressure of between 1 MPa and 10 MPa. The CO₂/ethylene partial pressure ratio is preferably between 1/10 and 10/1. More preferably, said ratio is between 2/8 and 8/2 and even more preferably between 2/1 and 1/2. The reaction temperature is preferably between 105° C. and 170° C., more preferably between 125° C. and 165° C., and more preferentially between 135° C. and 155° C.

### Separation Step d) (Optional)

At the end of the reaction, it is advantageous to proceed via a step of separating out the acrylate salt. In a preferential manner, a counter-solvent, immiscible with the reaction solvent, is added to the reaction medium and the whole is sent to a liquid-liquid separation step. In an even more preferential manner, the counter-solvent is water, potentially resulting partly from the dehydration of the ethanol in step b). At the outlet of the liquid-liquid separation, two streams are recovered, namely:
a first stream containing the reaction solvent and the catalytic precursor; and
a second stream, said to contain the counter-solvent enriched in acrylate salt and in the alcohol corresponding to the base used in the reaction.

The first stream is advantageously reinjected into the reactor, after any drying to remove traces of water, to recycle the catalyst and continue to produce acrylate salts.

The second stream is advantageously sent to another separation step, preferentially a distillation step, in which the counter-solvent, the alcohol and the acrylate salt are separated.

Advantageously, the stream enriched in counter-solvent may thus be recycled into the liquid-liquid extraction step.

Advantageously, the alcohol-enriched stream is sent to a base regeneration step. In this step, the alcohol is placed in contact with a strong base to reform the base, which is then introduced into the reactor. Preferentially, the strong base is sodium hydroxide and the water produced during the regeneration may optionally be used as a counter-solvent.

Depending on the use made thereof, the acrylate salt may either be recovered in the form of a concentrated solution in the counter-solvent, or isolated and purified by distillation, drying or crystallization methods known to those skilled in the art.

The acrylate salt may also be subsequently converted into acrylic acid by adding acid.

### EXAMPLES

1/ Case of the Synthesis of Sodium Acrylate from Glucose without Upgrading the CO₂ of the Fermentation Step (Non-Compliant):

The equation for the glucose fermentation reaction is C₆H₁₂O₆→2 C₂H₅OH+2 CO₂. As a result, 66.7% of the carbon in a glucose molecule is converted to ethanol and 33.3% to CO₂.

For a feedstock of 170 Kta of glucose, fermentation theoretically allows the production of 86.8 Kta of ethanol and 83 Kta of CO₂.

Assuming that the yield of ethanol dehydration into ethylene is 97%, it is possible to produce 51.2 Kta of ethylene, i.e. at best 172.1 Kta of sodium acrylate. Without CO₂ upgraded to sodium acrylate, up to 64.7% of the carbon in glucose may be upgraded to sodium acrylate (see Table 1 hereinbelow).

2/ Case of the Synthesis of Sodium Acrylate from Glucose with Upgrading of the CO₂ from the Fermentation Step (Compliant):

For a feedstock of 170 Kta of glucose, fermentation theoretically allows the production of 86.8 Kta of ethanol and 83 Kta of CO₂.

Assuming that the yield of ethanol dehydration into ethylene is 97%, it is possible to produce 51.2 Kta of ethylene, i.e. at best 172.1 Kta of sodium acrylate. To produce 172.1 Kta of sodium acrylate with 51.2 Kta of ethylene, at least 80.5 Kta of CO₂ are required. Fermentation thus produces enough CO₂ for the sequence to be self-sufficient in CO₂.

With the CO₂ upgraded to sodium acrylate by the sequence described in the invention, up to 97% of the carbon in the glucose may be upgraded to sodium acrylate (see Table 1 below).

TABLE 1

| | | % of C | % of C |
|---|---|---|---|
| | | upgradable into | upgradable into |
| Biomass | | acrylate without | acrylate with |
| upgrading | Production in | upgrading the CO₂ | upgrading of the |
| products | Kt/a | (non-compliant) | CO₂ (compliant) |
| Ethanol | 86.8 | 64.7% | 64.7% |
| CO₂ | 83.0 | | 32.3% |
| Total | | 64.7% | 97.0% |

1. A process for converting biomass into acrylate salts, successively comprising:
a) a step of treating the biomass to produce ethanol and carbon dioxide;
b) a step of dehydrating the ethanol obtained on conclusion of step a) to obtain ethylene;
c) a step of synthesizing acrylate salts from the ethylene obtained on conclusion of step b) and the carbon dioxide obtained on conclusion of step a) in the presence of a catalytic precursor and a solvent.

2. The process as claimed in claim 1, in which step a) comprises the following sub-steps:
a1) a step of pretreating biomass to obtain a pretreated substrate;
a2) a step of enzymatic or chemical hydrolysis of the pretreated substrate obtained on conclusion of step a1) to obtain an enzymatic or chemical hydrolysis must;
a3) a step of alcoholic fermentation of the enzymatic or chemical hydrolysis must obtained on conclusion of step a2) to obtain ethanol and carbon dioxide.

3. The process as claimed in claim 2, in which sub-step a1) is performed by steam explosion under acidic conditions at a temperature of between 150° C. and 250° C. and for a time of between 5 minutes and 30 minutes.

4. The process as claimed in claim 2, in which sub-step a2) is performed by enzymatic hydrolysis in the presence of Trichoderma reesei cellulases.

5. The process as claimed in claim 2, in which, when step a2) is an enzymatic hydrolysis, steps a2) and a3) are performed simultaneously.

6. The process as claimed in claim 1, in which step b) comprises the following sub-steps:
b1) a step of vaporizing a vaporization feedstock comprising the ethanol obtained on conclusion of step a) in a heat exchanger, said vaporization feedstock being introduced into said vaporization step at a pressure of between 0.1 MPa and 2.5 MPa so as to produce a vaporized feedstock;
b2) a step of superheating said vaporized feedstock obtained on conclusion of step b1) so as to bring said vaporized feedstock to an inlet temperature compatible with the dehydration reaction temperature;
b3) a step of dehydrating said feedstock obtained from step b2) in at least one adiabatic reactor containing at least one dehydration catalyst and in which the dehydration reaction takes place, operating at an inlet temperature of between 350° C. and 550° C. and at an inlet pressure of between 0.3 MPa and 1.8 MPa.

7. The process as claimed in claim 1, in which step c) is performed at a temperature of between 105° C. and 170° C., and at a pressure of between 1 MPa and 10 MPa.

8. The process as claimed in claim 1, in which step c) is performed in the presence of a base chosen from alkanolates of secondary or tertiary alcohols.

9. The process as claimed in claim 1, in which step c) is performed in the presence of a catalytic precursor based on a metal complex of a transition metal chosen from nickel (0) and palladium (0) complexes.

10. The process as claimed in claim 1, in which step c) is performed in the presence of a solvent chosen from anisole, cyclohexylpyrrolidone, N,N-dibutylformamide and dimethylacetamide.

11. The process as claimed in claim 1, also comprising a step d) of liquid-liquid separation of the acrylate salt in the presence of a counter-solvent which is immiscible with the solvent in step c).

12. The process as claimed in claim 11, in which said counter-solvent is water resulting at least partly from step b) of dehydration of the ethanol.

13. The process as claimed in claim 1, characterized in that the biomass is a lignocellulosic biomass.

14. A facility for converting biomass into acrylate salts that is capable of performing the process as claimed in claim 1, said facility comprising:
a first reaction section (2) allowing ethanol (3) and carbon dioxide (4) to be produced from biomass (1);
a second reaction section (5) for dehydration of the ethanol (3) into ethylene (6); and
a third reaction section (7) allowing acrylate salts (8) to be produced from ethylene (6) and carbon dioxide (4).
